(19) United States
(12) Patent Application Publication
(10) Pub. No.: US 2025/0331950 A1
(43) Pub. Date: Oct. 30, 2025

(54) DENTAL HANDPIECE HAVING AN IMPROVED PUSH BUTTON GUIDANCE

(71) Applicants: DENTSPLY SIRONA Inc., york, PA (US); SIRONA Dental Systems GmbH, Bensheim (DE)

(72) Inventor: Metin Ertugrul, Bensheim (DE)

(21) Appl. No.: 18/870,304

(22) PCT Filed: Jun. 28, 2023
(86) PCT No.: PCT/EP2023/067716

§ 371 Date: Nov. 27, 2024

(30) Foreign Application Priority Data
Jul. 27, 2022 (EP) 22187241.9
Sep. 14, 2022 (EP) 22195526.3

(51) Int. Cl. A61C 1/14

(52) U.S. Cl. CPC A61C 1/144; A61C 1/141

(57) ABSTRACT

The present invention relates to a dental handpiece (1) comprising: a head (4); a clamping means (13) which is rotatably arranged within a bearing (19) inside the head (4), and adapted for releasably clamping a dental drill (2), wherein the bearing (19) has an inner race (21) fixed to the clamping means (13) and an outer race (21) fixed to a housing (5) of the head (4); a driving means for rotating the clamping means (13) about an axial direction of the dental drill (2); a push button (7) which is arranged movably in the head (4) along the axial direction and configured to actuate the clamping means (13) to release the dental drill (2); and a biasing means to move the push button (7) away from the clamping means (13); characterized in that a radial inner surface (7a) of the push button (7) is arranged to contact and slide on a radial outer surface (20a) of the outer race (20) which configures a guide (12) for the moving push button (7), wherein the radial direction is perpendicular to axial direction of the dental drill (2); and wherein a radial clearance (A) is arranged between a radial inner surface (5a) of the housing (5) and a radial outer surface (7b) of the push button (7).

### TECHNICAL FIELD OF THE INVENTION

The present invention relates to dental handpieces, in particular to the push button guidance of a dental handpiece for actuating a clamping mechanism for a dental drill.

### BACKGROUND ART OF THE INVENTION

A dental handpiece is a handheld device for performing a variety of dental treatments which may require the use of various dental tools such as drills. The head of the dental handpiece is usually provided with a clamping mechanism such as a chuck that allows the dentist to change the dental tool in accordance with the intended dental treatment by means of a push button. The push button is movably held through a guide in the head of the dental handpiece.

Reference is made to EP0639354A2, EP 1733696A1, EP2959860A1, EP3412244 each disclosing a prior art dental handpiece.

There are certain design requirements that must be met by the dental handpiece to assure its proper operation. For example, any excessive heating of the push button at the contact area with the clamping mechanism must be prevented in as much as possible. For example, any excessive mechanical abrasion of the clamping jaws must be prevented in as much as possible. For example, the size of the head should be reduced as much as possible so that it can easily reach spatially limited places in the oral cavity. For example, the push button and the clamping mechanism must be operable in a reliable and easy manner.

### DISCLOSURE OF THE INVENTION

The inventor is not aware of any prior art handpiece in which the radial inner surface of the push button is arranged to directly contact and slide on the radial outer surface of the outer race of the bearing which rotatably holds the burr shaft. Thereby, the burr shaft axis and the push button axis can be precisely aligned, and thus the push button can be operated highly smoothly.

The objective of the present invention is to overcome one or more of the disadvantages of the prior art and provide a dental handpiece which is mechanically compact and easy to use.

This objective has been achieved through the dental handpiece as defined in claim 1.

The dental handpiece of the present invention comprises: a head; a clamping means which is rotatably arranged within a bearing inside the head, and adapted for releasably clamping a dental drill, wherein the bearing has an inner race fixed to the clamping means and an outer race fixed to a housing of the head; a driving means for rotating the clamping means about an axial direction of the dental drill; a push button which is arranged movably in the head along the axial direction and configured to actuate the clamping means to release the dental drill; and a biasing means to move the push button away from the clamping means. The radial inner surface of the push button is arranged to contact and slide on the radial outer surface of the outer race which configures a guide for the moving push button along the axial direction, wherein the radial direction is perpendicular to axial direction of the dental drill. A radial clearance is arranged between the radial inner surface of the housing and a radial outer surface of the push button.

Another major advantageous effect of the present invention is that the size of the head can be reduced. This can be explained as follows. Since the radial outer surface of the outer race which configures a guide for the moving push button along the axial direction, thereby the need for providing any separate guidance member can be avoided. This allows a compact mechanical configuration. The compact size of the head will also allow the dentist to easily reach spatially limited places in the oral cavity and to work more easily inside the oral cavity.

Another major advantageous effect of the present invention is that the push button is operable in a more reliable and easy manner. This can be explained as follows: Since the radial outer surface of the outer race which configures a guide for the moving push button along the axial direction, the motion can be property aligned with the bearing surfaces, and thereby with the drill of the clamping mechanism. This enables a good alignment between the moving parts.

In alternative embodiments of the present invention the push button can be provided as a single piece member or a two-piece member.

### BRIEF DESCRIPTION OF THE DRAWINGS

In the subsequent description, the present invention will be described in more detail by using exemplary embodiments and by referring to the drawings, wherein

FIG. 1—is a schematic partial side view of a dental handpiece according to an embodiment of the invention;

FIG. 2a—is a schematic partial vertical cross-sectional enlarged view of the head of the dental handpiece in FIG. 1, taken along the line I-I;

FIG. 2b—is a schematic partial cross-sectional enlarged view of the head of the dental handpiece in FIG. 1, taken along the line I-I in the actuated state;

FIG. 2c—is a schematic partial cross-sectional view of the head of the dental handpiece of FIG. 1, taken along the line II-II;

FIG. 2d—is a schematic partial cross-sectional enlarged view of the head of a dental handpiece according to an alternative embodiment of the invention, taken along the line II-II in FIG. 1;

FIG. 3—is a schematic partial cross-sectional enlarged view of the clamping mechanism inside the bearings of the dental handpiece in FIG. 1, taken along the line I-I;

FIG. 4a—is a schematic partial cross-sectional enlarged view of the clamping mechanism of FIG. 3 without the bearings;

FIG. 4b—is a schematic partial cross-sectional view of the clamping mechanism of FIG. 4a, in the actuated state;

FIG. 5a—is a schematic side view of the chuck of the dental handpiece in FIG. 1;

FIG. 5b—is a schematic perspective view of the chuck of the dental handpiece in FIG. 1;

FIG. 6a—is a schematic perspective view of the flange collar (hat-shaped) of the dental handpiece in FIG. 1;

FIG. 6b—is a schematic perspective view of the outer race and the inner race of the dental handpiece in FIG. 1;

FIG. 6c—is a schematic perspective view of the cap of the dental handpiece in FIG. 1;

FIG. 6d—is a schematic perspective view of the screw ring of the dental handpiece in FIG. 1;

FIG. 7a—is a schematic vertical cross sectional view of the two-piece push button of the dental handpiece in FIG. 2c;

FIG. 7b—is a schematic perspective view of the two-piece push button in FIG. 7a;

FIG. 7c—is a schematic vertical cross sectional view of the single-piece push button of the dental handpiece in FIG. 2d;

FIG. 7d—is a schematic perspective view of the single-piece push button in FIG. 7c;

The reference numbers shown in the drawings denote the elements as listed below and will be referred to in the subsequent description of the exemplary embodiments.
1. Dental handpiece
2. Dental drill (burr)
3. Body
4. Head
5. Housing
5a. Gap
5b. Radial inner surface
6. Internal Thread
7. Pushbutton
7a. Radial inner surface
7b. Radial outer surface
8. Cap
9. Flange collar (hat-shaped)
9a. Claw
9b. Air Gap (thermal insulation)
9′ Cap
10. Contact surface (point like)
11. Spring (cone shaped)
12. Guide
13. Clamping means
14. Ball
15. Hollow shaft
16. Gear
17. Opening
18. Stop Edge
19. 19′ Bearing
20. Outer race
20a. Radial outer surface
21. Inner race
22. Balls
23. Retainer
24. Screw ring
25. Nose
26. Chuck
27. Arm
27a. Contact point
28. Cut-out
29. Large diameter portion
30. Small diameter portion
31. Contact edge (Burr stop)
32. Driving shaft
33. Bearing
34. Outer race
35. Inner race
36. Balls
37. Retainer
38. Crown gear

### A: Axial Direction

### R: Radial Direction

### S: Radial Clearance

### F: Actuation Force

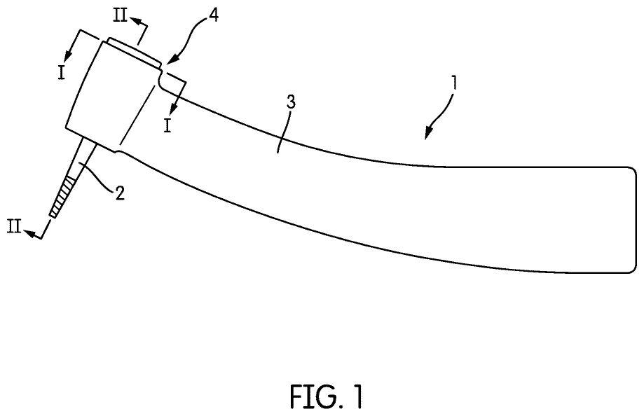

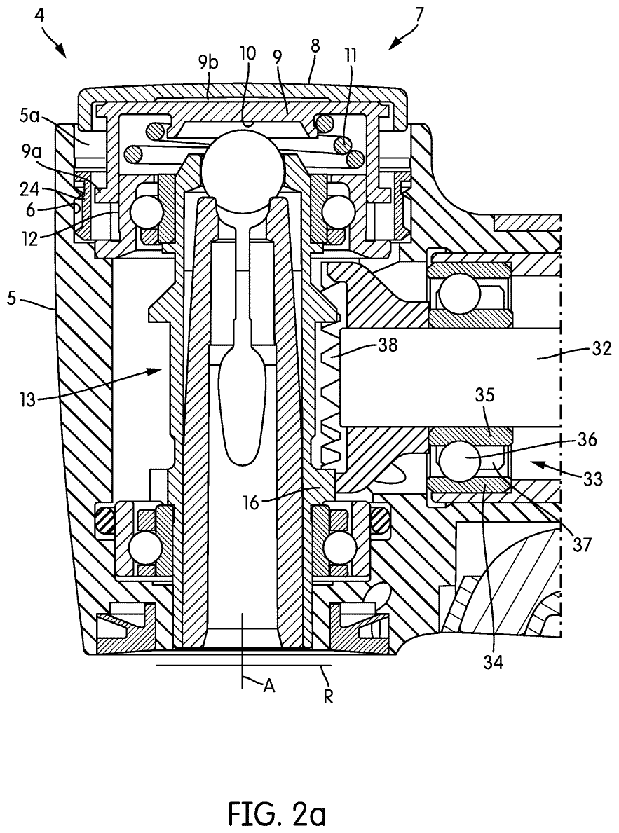

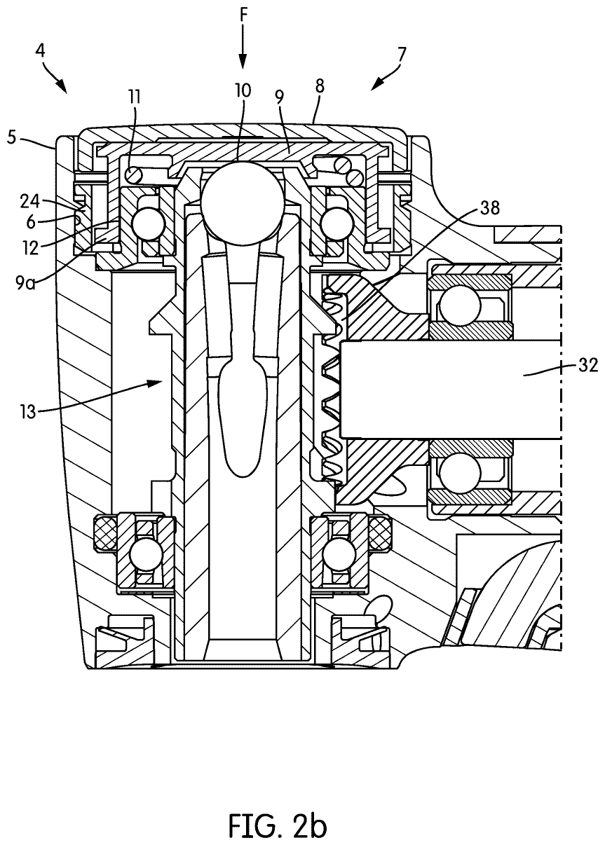

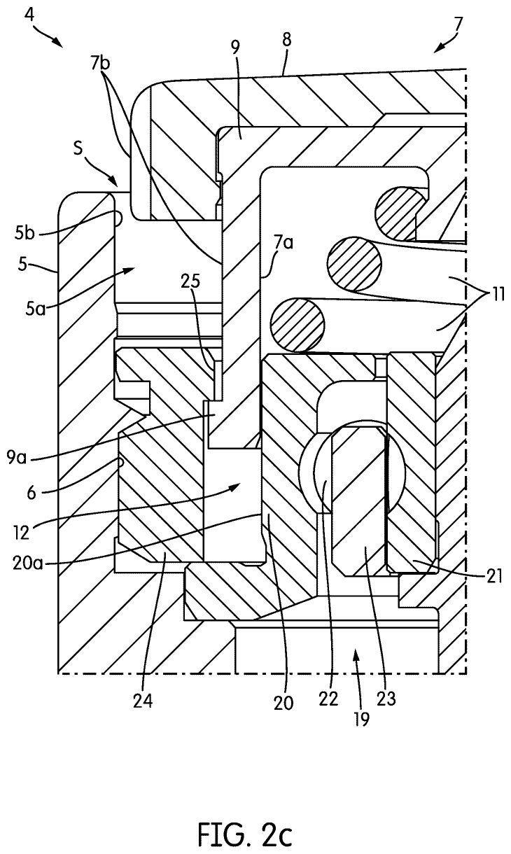

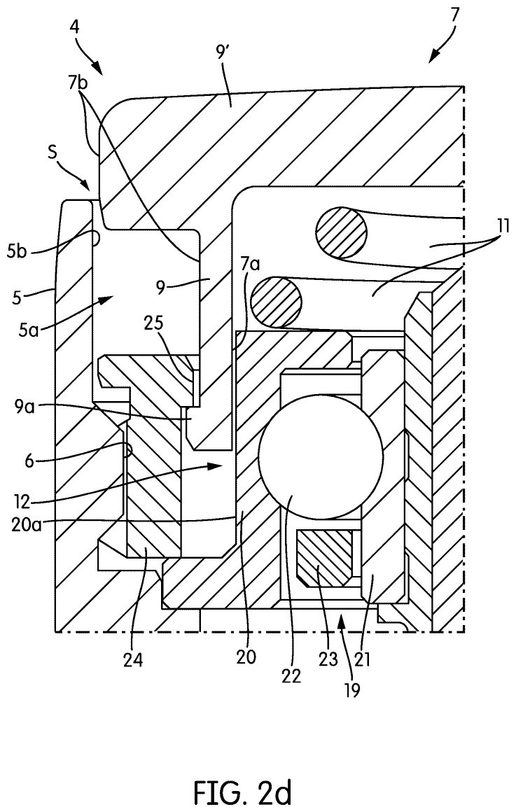

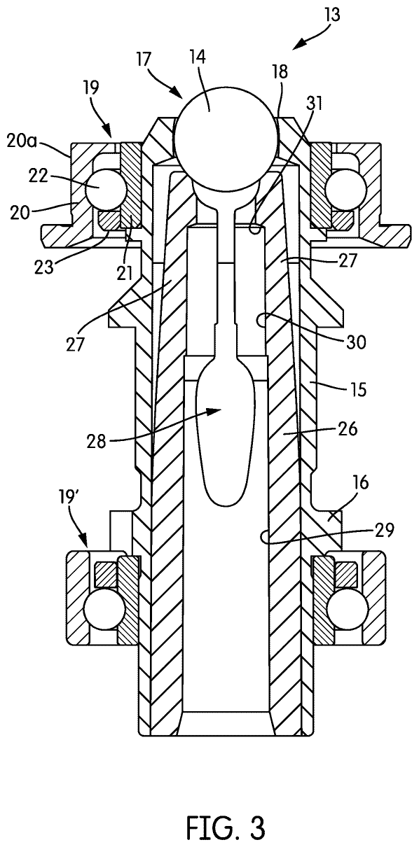

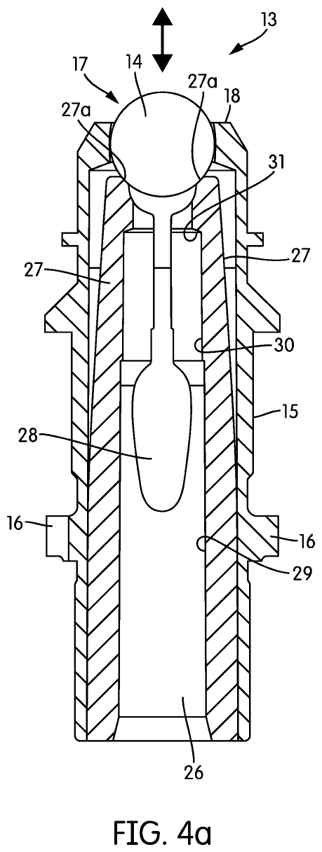

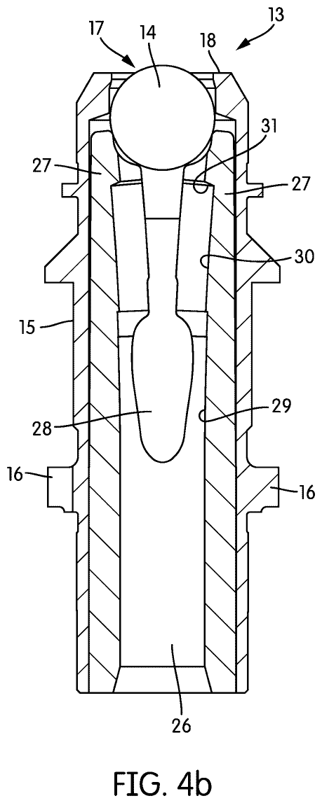

FIG. 1 shows a dental handpiece (1) according to an embodiment of the invention. As shown in FIG. 1, dental handpiece (1) has a body (3) and a head (4). A dental drill (2) is detachably attached to the head (4). FIG. 2a shows an enlarged cross-sectional view of the head (4). As shown in FIG. 2a, the head (4) has a housing (5) which accommodates a clamping means (13). As shown in FIG. 4a the clamping means (13) has a chuck (26) with resilient arms (27) for releasably clamping the dental drill (2). A hollow shaft (15) is rotatably arranged inside the head (4). The chuck (26) is fixed into the hollow shaft (15) to rotate with same. The dental handpiece (1) has a driving means such as a motor (not shown) for rotating the hollow shaft (15). The driving means is coupled to a driving shaft (32) as shown in FIG. 2a, that is rotatably held through a bearing (33) having an outer race (34), an inner race (35), multiple balls (36), and a retainer (37). The end of the driving shaft (32) has a crown gear (38) that meshes with a gear (16) on the hollow shaft (15). As shown in FIG. 3 the hollow shaft (15) is rotatably held through a couple of bearings (19; 19′) mounted into the housing (5). Each bearing (19;19′) has an outer race (20) and inner race (21), multiple balls (22) and a retainer (23).

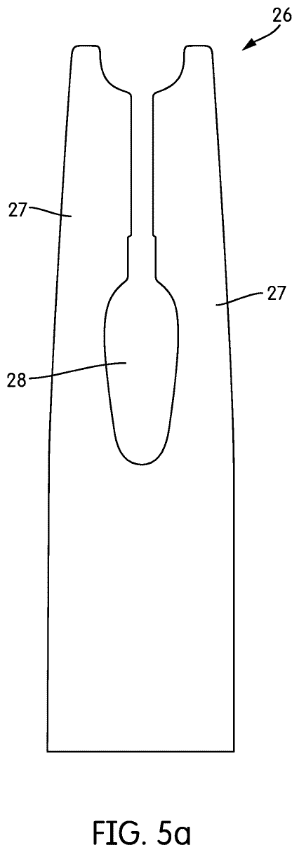

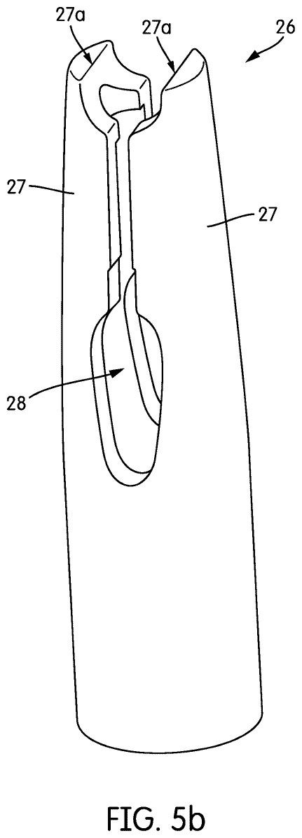

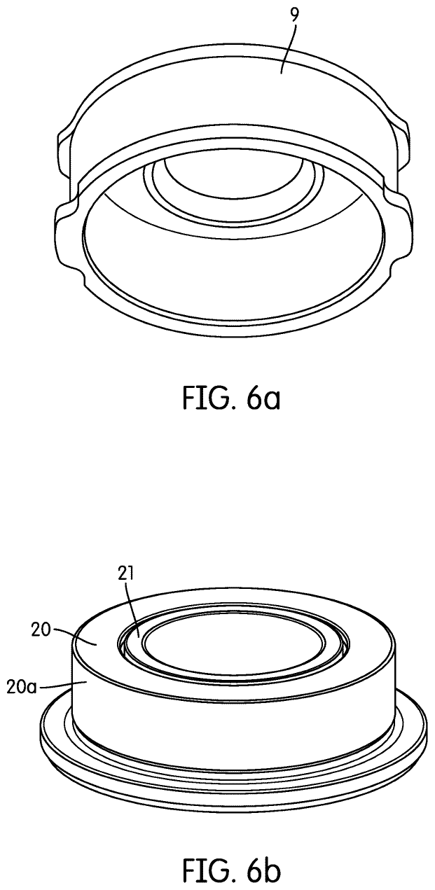

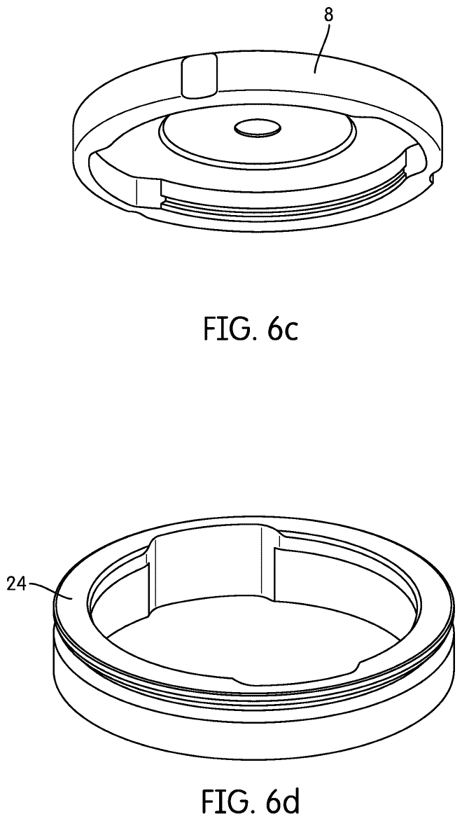

As shown in FIG. 3, the clamping means (13) has a ball (14), preferably made of ceramic material, which is axially movably arranged inside the rotatable hollow shaft (15) and in direct contact with the resilient arms (27) of the chuck (26). The ball (14) partly protrudes through an opening (17) in the upper end of the hollow shaft (15). The size of the opening (17) is smaller than the size of the ball (14) so that it is retained partly within the hollow shaft (15). As shown in FIG. 3 the opening (17) has an annular stop edge (18) formed integrally with the upper end of the hollow shaft (15), which restricts the outwards movement of that ball (14). As shown in FIG. 2a and FIG. 2b the dental handpiece (1) has a push button (7) which is arranged in the head (4) and configured to contact, when pressed by a user, the partly protruding ball (14) and move the ball (14) downwardly in between the resilient arms (27) to flex the resilient arms (27) radially outwards and release the dental drill (2). As shown in FIG. 5a the chuck (26) has two diametrically opposed cut-outs (28) in its upper portion to form the two resilient arms (27). As shown in FIG. 5b, a linear semi cylindrical channel is formed into the upper portion of the chuck (26) whose edges form two diametrically opposed contact points (27a) to directly contact the ball (14). As shown in FIG. 3, the chuck (26) has a large diameter portion (29) and a small diameter portion (30). When the resilient arms (27) are retracted, the dental drill (2) can be manually inserted only until the upper end of the large diameter portion (29). When the resilient arms (27) are flexed through the ball (14), the dental drill (2) can be further inserted into the small diameter portion (30) until it abuts against the contact edge (31) (Burr stop). Once the push button (7) is released, the resilient arms (27) are retracted, and the dental drill (2) is clamped in the small diameter portion (30) between the resilient arms (27). The resilient arms (27) also bias the ball (14) inside the rotatable hollow shaft (15) towards the opening (17). As shown in FIG. 2a, the pushbutton (7) has a cap (8), and a hat-shaped flange collar (9) arranged inside the cap (8) and fixed thereto. The hat-shaped flange collar (9) has a point like contact surface (10), preferably planar, for directly contacting the ball (14). The pushbutton (7) has a biasing means such a spring (11), preferably a cone shaped spring (11) to move the push button (7) away and detach it from the ball (14). As shown in FIG. 2a, the point like contact surface (10) is not in contact with the ball (14) when the push button (7) is released. As shown in FIG. 2b, the contact surface (10) comes in point contact with the upper side of the ball (14) when the push button (7) is pressed downwards.

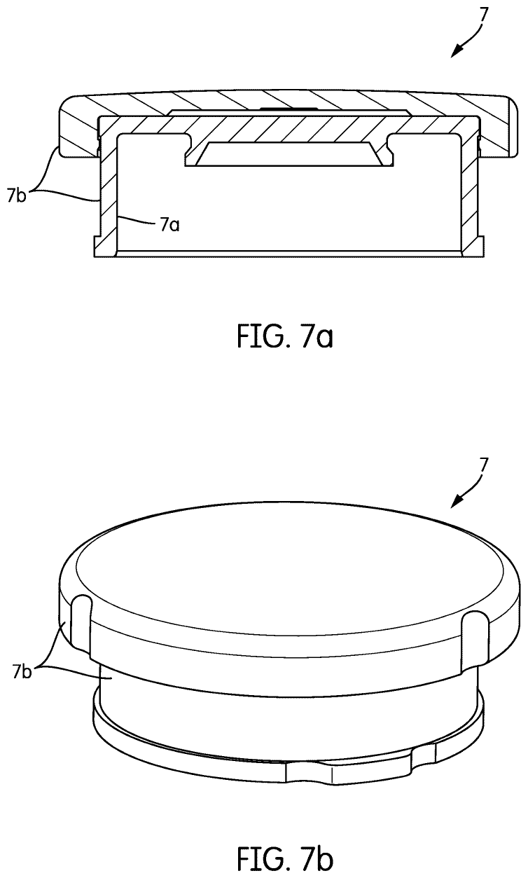

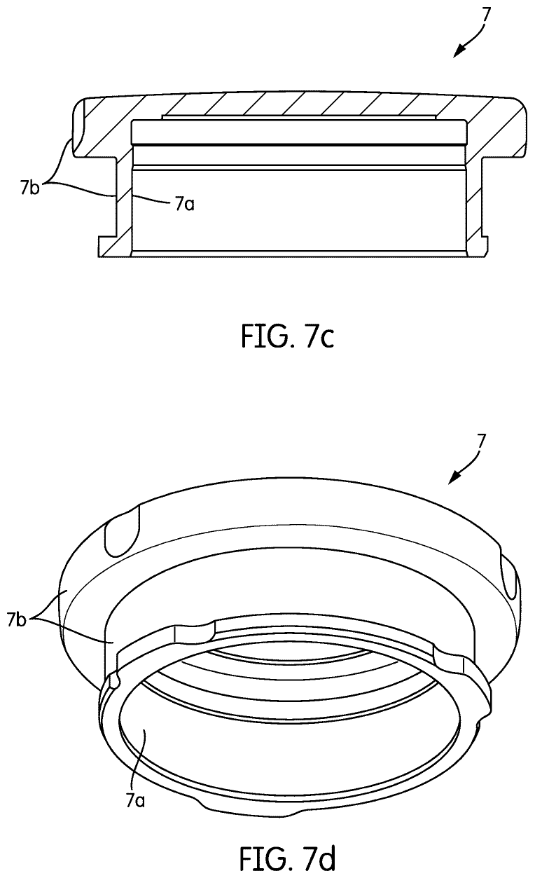

FIG. 2c shows an enlarged view of the two-piece pushbutton (7) in FIG. 2a which is configured by the cap (8) and the hat-shaped Flange collar (9). FIG. 7a and FIG. 7b, show the two-piece push button (7) in more detail.

FIG. 2d shows an alternative embodiment, wherein the cap (9′) is provided as a single piece instead of two separate pieces. All other features remain the same. Thereby the number of pieces can be further reduced. FIGS. 7c and 7d, show the single-piece push button (7) in more detail.

According to the alternative embodiments as shown in FIGS. 2c and 2d, a radial inner surface (7a) of the push button (7) is arranged to directly contact and slide on a radial outer surface (20a) of the outer race (20) which configures a guide (12) for the moving push button (7). The radial direction is perpendicular to the axial direction (A) of the dental drill (2). A radial clearance (s) is arranged between a radial inner surface (5b) of the housing (5) and a radial outer surface (7b) of the push button (7). The housing (5) is formed from a single piece.

As shown in FIG. 2a, the housing (5) has an internal thread (6) which receives a separate screw ring (24). A mounting flange of the upper bearing (19) is fixed to its mounting position through the screw ring (24). The mounting flange has an L-shaped cross section and is integrally formed with the outer race (20) of the bearing (19). The screw ring (24) has a nose (25) which can be seen in FIG. 2d but cannot be seen in FIG. 2a. The cross-sectional views respectively in FIG. 2a and FIG. 2d are rotated about 90 degrees with respect to each other. The cross-section in FIG. 2a is along the line I-I as shown in FIG. 1 and the cross section in FIG. 2d is along the line II-II as shown in FIG. 1. As shown in FIG. 2d, the nose (25) extends radially inwards and can abut from below against the claw (9a) of the hat-shaped flange collar (9) which extends radially outwards and prevents the push button (7) from disengaging from the screw ring (24), and thus from the housing (5). The hat-shaped flange collar (9) can be provided separately from or integrally with the cap (8;9′). In the former case an air gap (9b) is preferably formed underneath the cap (8) to improve thermal insulation as shown in FIG. 2a. Alternatively the air gap (9b) may be filled with a material that is a poor heat-conductor.

As shown in FIG. 2a and FIG. 2d, the inner surface of the hat-shaped flange collar (9) slides directly on the outer surface of outer race (20) while being in contact with the latter which configures a guide (12) for the push button (7) movement. The nose (25) of the screw ring (24) is arranged above the claw (9a) of the hat-shaped flange collar (9). The hat-shaped flange collar (9) does not contact the inner radial surface of the screw ring (24). The flange collar (9) is inserted between the screw ring (24) and the outer race (20). The housing (5) has a gap (5a) directly above the screw ring (24) and the nose (25), which allows the cap (8;9′) to move upwards or downwards during the clamping/releasing operation. The screw ring (24) is entirely embedded/recessed inside the housing (5) and does not project outside the head (4). Thereby, the height of the head (4) can be reduced. Thereby also the diameter of the cap (8,9′) can be increased so that the actuating pressure on the finger is reduced. The outer cylindrical surface of the cap (8;9′) does not contact the inner surface of the housing (5) during its entire movement thanks to the sufficient radial clearance (S). Thus, the guide (12) serves as a guiding contact surface for the up/down movement of the cap (8;9′). Through the above described configuration, the height and diameter of the head (4) can be further reduced.

1. A dental handpiece comprising:
a head;
a clamping means which is rotatably arranged within a bearing inside the head, and adapted for releasably clamping a dental drill, wherein the bearing has an inner race fixed to the clamping means and an outer race fixed to a housing of the head;
a driving means for rotating the clamping means about an axial direction of the dental drill;
a push button which is arranged movably in the head along the axial direction and configured to actuate the clamping means to release the dental drill; and
a biasing means to move the push button away from the clamping means;
wherein a radial inner surface of the push button is arranged to contact and slide on a radial outer surface of the outer race which configures a guide for the moving push button, wherein a radial direction is perpendicular to the axial direction of the dental drill; and wherein a radial clearance is arranged between a radial inner surface of the housing and a radial outer surface of the push button.

2. A dental handpiece comprising:
a head;
a clamping member rotatably arranged within a bearing inside the head, the clamping member configured to releasably clamp a dental drill;
a driving member configured to rotate the clamping member about an axial direction of the dental drill;
a push button disposed at least partially in the head and movable along the axial direction, the push button configured to actuate the clamping member to release the dental drill; and
a biasing member configured to move the push button away from the clamping member.

3. The dental handpiece of claim 2, wherein the bearing has an inner race and an outer race.

4. The dental handpiece of claim 3, wherein the inner race is fixed to the clamping member.

5. The dental handpiece of claim 4, wherein the outer race is fixed to a housing of the head.

6. The dental handpiece of claim 5, wherein the push button includes a radial inner surface configured to contact and slide on a radial outer surface of the outer race, thereby providing a guide for moving the push button.

7. The dental handpiece of claim 6, wherein a radial clearance is arranged between a radial inner surface of the housing and a radial outer surface of the push button.

8. The dental handpiece of claim 2, further comprising a guide for moving the push button along the axial direction.

9. The dental handpiece of claim 2, wherein the biasing member comprises a spring.

10. The dental handpiece of claim 9, wherein the spring is cone shaped.

11. The dental handpiece of claim 2, wherein the clamping member includes a chuck with resilient arms configured to releasably clamp the dental drill.

12. The dental handpiece of claim 11, wherein a hollow shaft is rotatably arranged inside the head.

13. The dental handpiece of claim 12, wherein the chuck is fixed into the hollow shaft for rotation therewith.

14. The dental handpiece of claim 13, wherein the clamping member includes a ball that is axially and movably arranged inside the hollow shaft.

15. The dental handpiece of claim 14, wherein the ball is formed from a ceramic material.

16. The dental handpiece of claim 14, wherein the ball is in direct contact with the resilient arms of the chuck.

17. The dental handpiece of claim 16, wherein the ball partly protrudes through an opening in an upper end of the hollow shaft.

18. The dental handpiece of claim 2, wherein the push button is a single-piece push button.

19. The dental handpiece of claim 2, wherein the push button is a two-piece push button.

20. The dental handpiece of claim 19, wherein the push button includes a cap and a hat-shaped flange collar.
